(19) United States
(12) Patent Application Publication
(10) Pub. No.: US 2014/0200503 A1
(43) Pub. Date: Jul. 17, 2014

(54) SYSTEMS, DEVICES, AND METHODS FOR ISOLATION OF STEM CELLS

(71) Applicants: Patricio CENTURION, Lima (PE); Adrián NORIEGA, Lima (PE)

(72) Inventors: Patricio CENTURION, Lima (PE); Adrián NORIEGA, Lima (PE)

(21) Appl. No.: 13/744,061

(22) Filed: Jan. 17, 2013

(51) Int. Cl. A61N 5/06; A61M 1/00

(52) U.S. Cl. CPC A61N 5/062; A61M 1/008; A61N 5/0625
USPC 604/21; 604/20; 607/89

(57) ABSTRACT

The present disclosure relates, in some embodiments, to apparatus, systems, methods, and compositions for purifying cells (e.g., adipocytes, stem cells) from mammalian tissues (e.g., adipose tissues). For example, the present disclosure relates to apparatus, systems, methods, and compositions for purifying and/or preserving adipocytes and/or stem cells from fat tissue of a mammalian subject according to some embodiments.

### FIELD OF THE DISCLOSURE

The present disclosure relates, in some embodiments, to apparatus, systems, methods, and compositions for harvesting cells (adipocytes, stem cells, fibroblasts) from subcutaeous tissues (e.g., human subcutaeous tissues). The present disclosure relates to apparatus, systems, methods, and compositions for harvesting and/or preserving adipocytes and/or stem cells from the fat tissue of a human subject according to some embodiments.

### BACKGROUND OF THE DISCLOSURE

Autologous fat grafting is a technique shown to be beneficial as a reconstructive and cosmetic procedure for patients with volume loss due to disease, trauma, congenital defects, or the natural process of aging. Adipose-derived mesenchymal stem cells (ASCs) have been identified as an ideal source of cells for regenerative medicine with the potential to differentiate into a variety of cell lineages for tissue engineering.

The standard aspirated subcutaneous tissue is composed of mature adipocytes, extracellular matrix, ASCs, endothelial cells, and mural cells (pericytes and vascular smooth muscle cells). When enzymatically digested, the non-buoyant cellular fraction forms the stromal vascular fraction (SVF) and contains ASCs, vascular progenitor cells, pericytes, and endothelial cells.

### SUMMARY

Accordingly, a need has arisen for improved apparatus, systems, methods, and compositions for purifying cells (e.g., adipocytes, stem cells) from mammalian tissues (e.g., adipose tissues). The present disclosure relates, according to some embodiments, to apparatus, systems, methods, and compositions for harvesting and/or preserving adipocytes and/or stem cells from fat tissue of a mammalian subject according to some embodiments. For example, a method for isolating cells (e.g., adipocytes and/or stem cells) from a subject (e.g., a human subject) may include (a) contacting a deep layer of tissue with an aqueous infiltration fluid comprising saline and at least one pharmachologically active catecholamine prechilled to a temperature below 10° C. to form an infiltrated tissue, (b) illuminating a target site in the infiltrated tissue with light at a wavelength from about 1150-nm to about 1800-nm to produce at least a volume of liquified tissue comprising cells, and/or (c) aspirating at least some of the volume of liquified tissue comprising cells. Illuminating a target site may comprise, according to some embodiments, illuminating a target site with light at a wavelength of about and/or exactly 1210-nm. In some embodiments, a method may include monitoring the temperature of the target site. A method may include, according to some embodiments, regulating the temperature of the target site. For example, a target site may be kept at a temperature from about 27° C. to about 33° C.

The present disclosure relates, in some embodiments, to systems for isolating cells (e.g., adipocytes and/or stem cells) from a subject (e.g., a human subject). A system may include, for example, a diode laser of 1210-nm wavelength, a delivery cannula in optic communication with the diode laser and configured to illuminate a target site in a deep layer of the subject, a fluid removal cannula configured to be positioned near the target site, a fluid receptical in fluid communication with the fluid removal cannula, and/or an aspiration pump operably coupled to the fluid removal cannula.

In some embodiments, the present disclosure relates to methods for harvesting adipocyte stem cells and adipocytes from a first subject for administration to a second subject. A method may comprise, for example, contacting a deep layer of tissue with an aqueous infiltration fluid comprising saline and at least one pharmachologically active catecholamine prechilled to a temperature below 10° C. to form an infiltrated tissue, illuminating a target site in the infiltrated tissue with light at a wavelength from about 1150-nm to about 1800-nm to produce at least a volume of liquified tissue comprising cells, and/or aspirating at least some of the volume of liquified tissue comprising cells, wherein the aspirated volume of liquified tissue comprises adipocyte stem cells and adipocytes. In some embodiments, a method may include contacting a second subject actually or potentially in need of fat grafting or a regenerative medicine procedure with at least a portion of the aspirated volume of liquified tissue comprising adipocyte stem cells and adipocytes.

The present disclosure relates, in some embodiments, to methods for selective photothermostimulation of a target site (e.g., a subcutanous target site). A method may include, for example, illuminating a subcutaneous target site with light from a diode laser at a wavelength of about 1210-nm through an optic fiber of about 600 μm, wherein mitochondrial activity, cell proliferation or mitochondrial activity and cell proliferation in the illuminated subcutaneous target site is increased.

### BRIEF DESCRIPTION OF THE DRAWINGS

Some embodiments of the disclosure may be understood by referring, in part, to the present disclosure and the accompanying drawings, wherein:

FIG. 1 illustrates the anatomy and layers between the epidermis and muscle of a treatment area before being exposed to the laser according to an embodiment of the disclosure;

FIG. 2 illustrates infiltration of tissue to be treated with infiltrated solution, which is being applied in the deep layer, under the hypodermis according to an embodiment of the disclosure;

FIG. 3 illustrates a laser application according to an embodiment of the disclosure, beginning in the deep layer and going upward toward the superficial layer;

FIG. 4 illustrates aspiration of treated adipose tissue according to an embodiment of the disclosure beginning in the deep layer with slow movements upward until reaching the superficial layer.

FIG. 5 is an ultrasound image illustrating an infiltration process according to an embodiment of the disclosure showing the infiltration process between subcutaneous tissue and muscle;

FIG. 6 is an ultrasound image illustrating an area after laser treatment, according to an embodiment of the disclosure, showing the condition of the layers of the treated area;

FIG. 7 is an ultrasound image illustrating an aspiration process in the subcutaneous tissue according to an embodiment of the disclosure showing the reduction in the volume of the layers of the area;

FIG. 8A illustrates fat aspirated according to an embodiment of the disclosure in stored in a sterile glass container for laboratory processing;

FIG. 8B illustrates fat aspirated by a conventional liposuction technique stored in a sterile glass container for laboratory processing;

FIG. 9 illustrating fat aspirated by laser liposuction according to an embodiment of the disclosure after centrifugation;

FIG. 10 is a flowchart illustrating an adipocyte harvesting process according to an embodiment of the disclosure;

FIG. 11A is a micrograph imaged at 20× showing adipose tissue obtained from lipoaspirate samples that underwent treatment with Laser 1210-nm according to an example embodiment of the disclosure;

FIG. 11B is a micrograph imaged at 40× showing adipose tissue obtained from lipoaspirate samples that underwent treatment with Laser 1210-nm according to an example embodiment of the disclosure;

FIG. 12A is a micrograph imaged at 20× showing adipose tissue obtained from lipoaspirate samples that underwent conventional liposuction treatment;

FIG. 12B is a micrograph imaged at 40× showing adipose tissue obtained from lipoaspirate samples that underwent conventional liposuction treatment, and

FIG. 13 illustrates a curved cannula according to an example embodiment of the disclosure, both along the plane of curvature (left) and 90° from the plane of curvature (right).

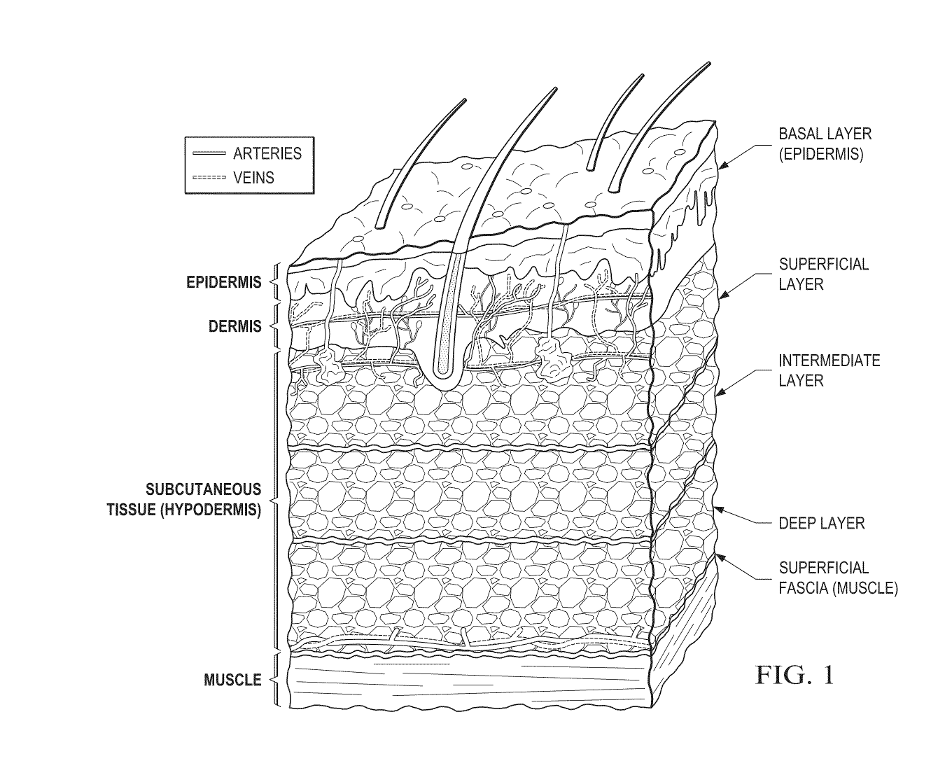

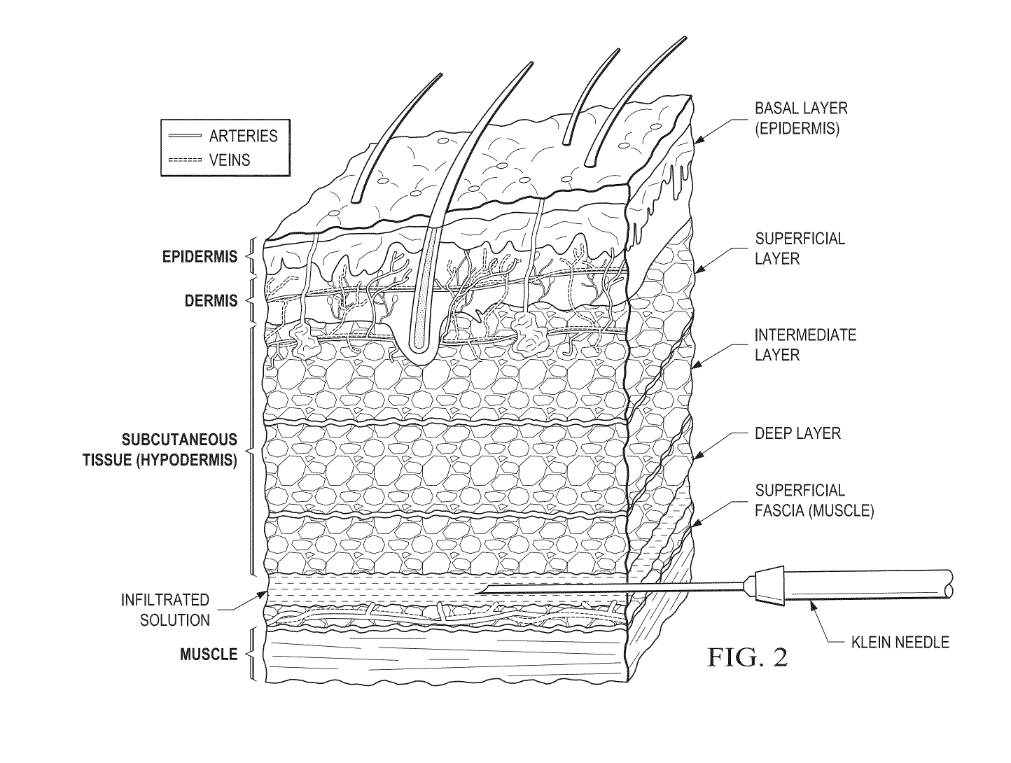

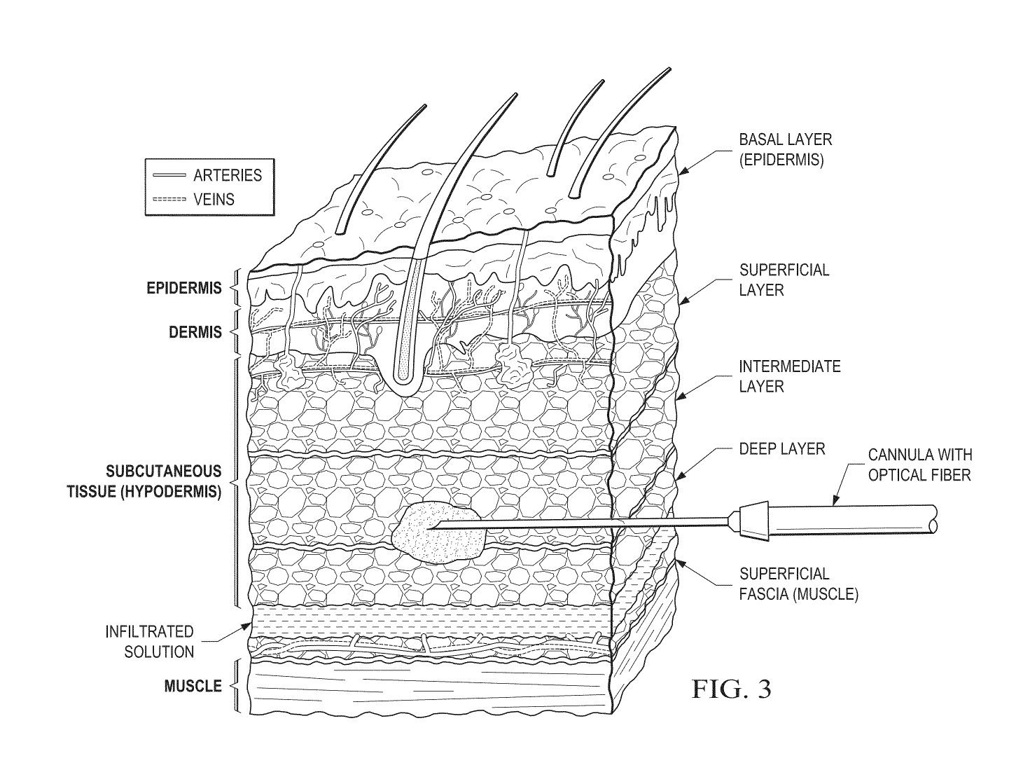

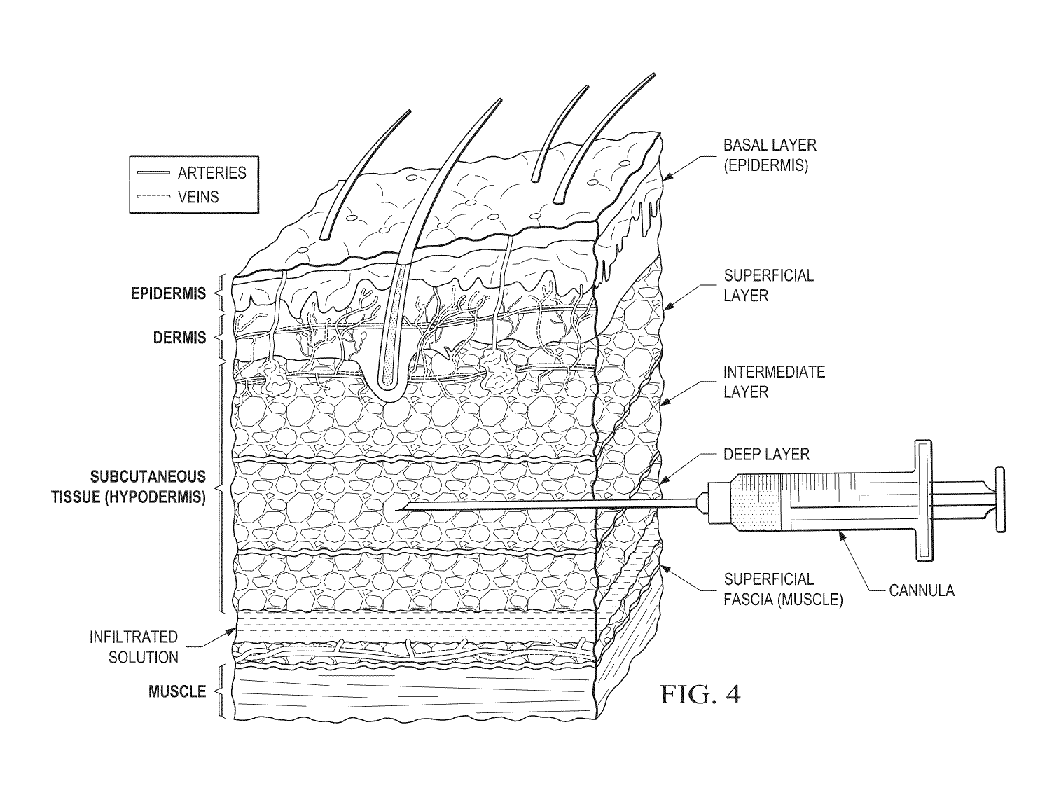

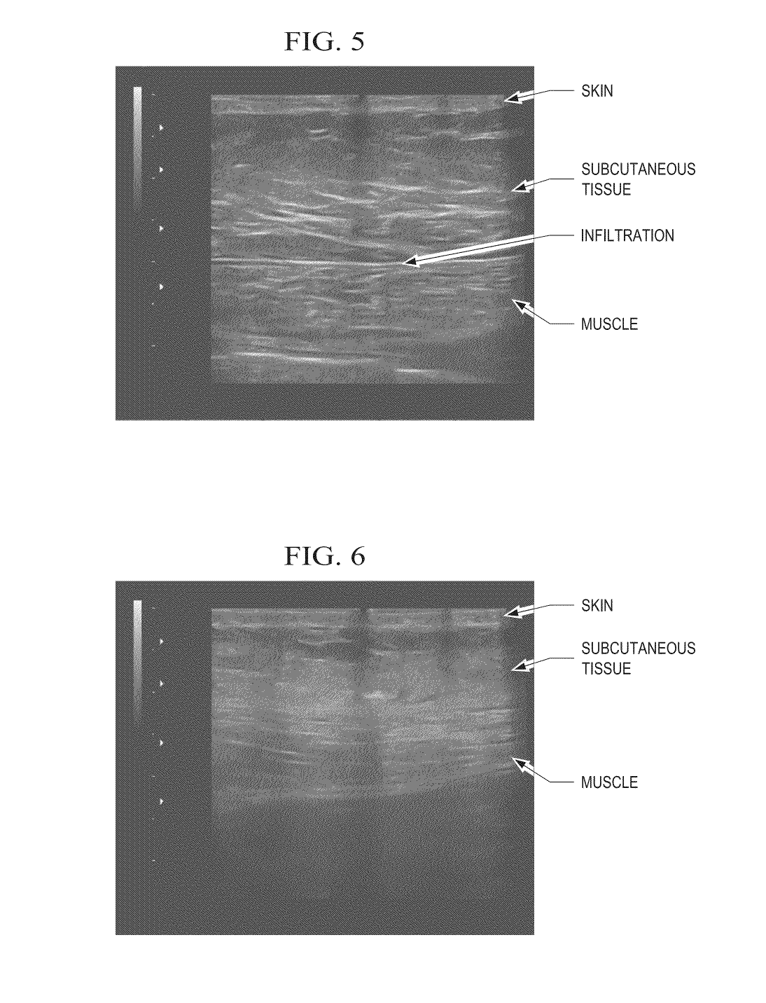

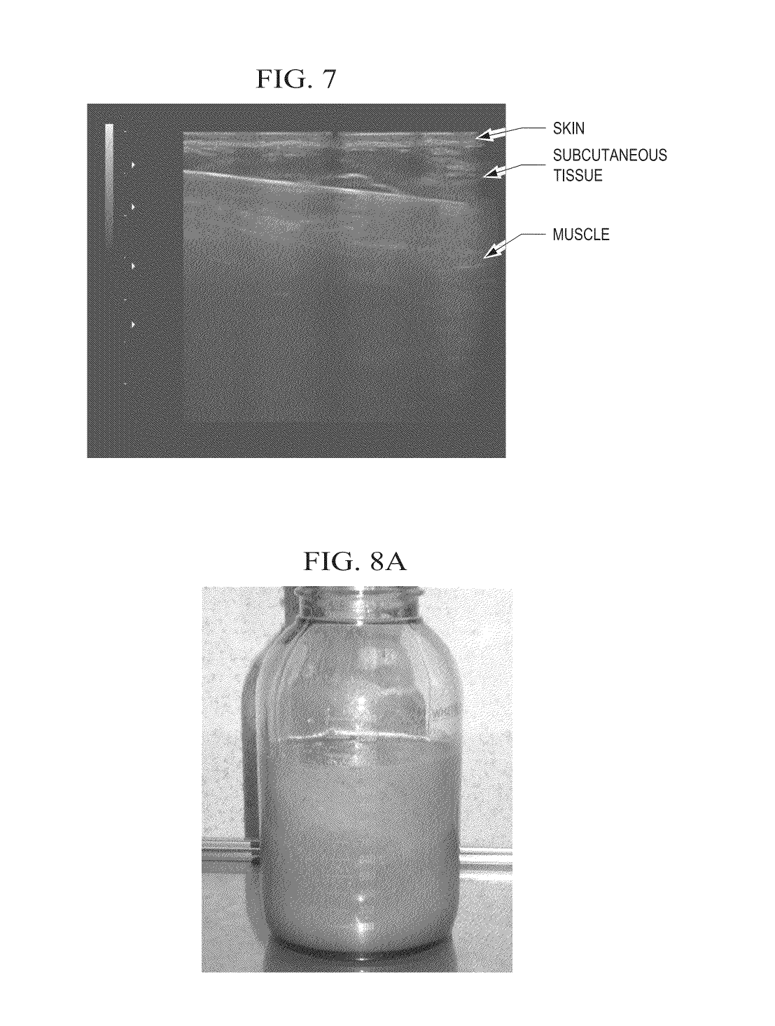

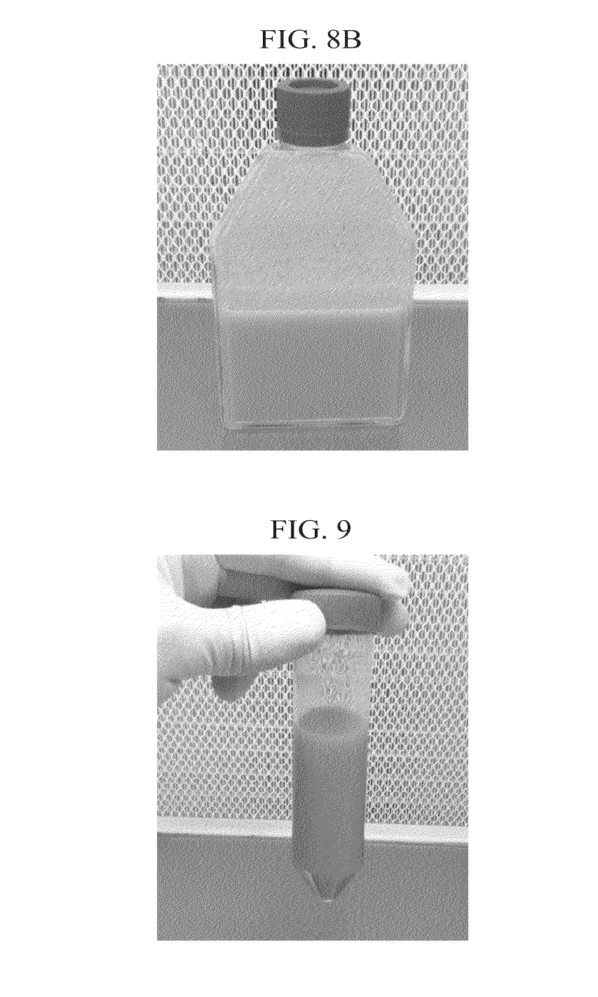

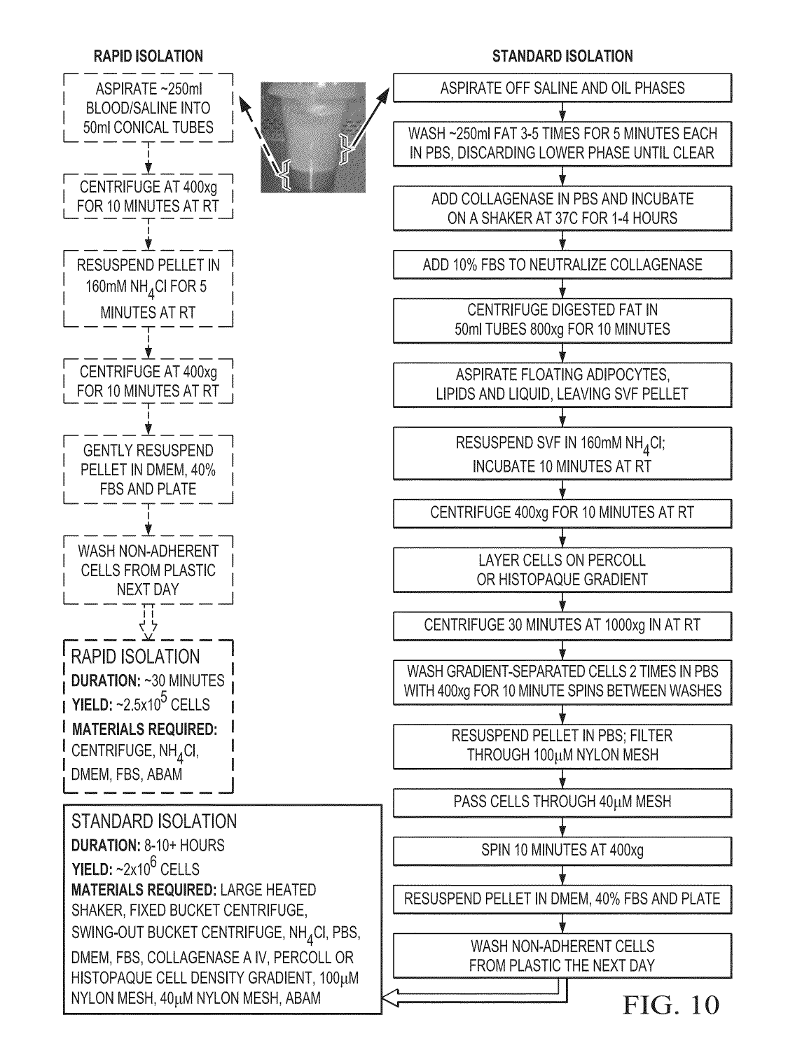

### DETAILED DESCRIPTION

The present disclosure relates, in some embodiments, to apparatus, systems, methods, and compositions for harvesting cells (e.g., adipocytes, stem cells, fibroblasts) from subcutaeous tissues (e.g., human subcutaeous tissues). The present disclosure relates to apparatus, systems, methods, and compositions for harvesting and/or preserving adipocytes and/or stem cells from fat tissue of a human subject according to some embodiments.

Since there is no available technique that preserves the integrity of the fat tissue and its stem cells, we initiated our research searching for a gold standard that offers less trauma for the patient, less damage and high preservation of the subcutaneous tissue and its adipose and stem cells.

According to some embodiments, the present disclosure provides diode laser-assisted liposuction (LSDL 1210-nm) methods, apparatus, and compositions. Cells (e.g., fat cells) may be harvested ad integrum in some embodiments. Methods of harvesting fat cells according to some embodiments may comprise “disruption” of adipose tissue rather than lipolysis. Fat cells harvested according to such methods may be used for fat grafting in liposculpture or breast reconstruction. Methods according to some embodiments of the disclosure may display a low complication rate, due to the affinity of this wavelength (1210-nm) with adipose tissue.

### Compositions

The present disclosure relates, in some embodiments, to compositions for harvesting cells (e.g., adipocytes, stem cells, fibroblasts) from subcutaeous tissues (e.g., human subcutaeous tissues). An example of a composition is an infiltration solution, which may be delivered (e.g., injected) into a subject. An infiltration solution may comprise, for example, adrenaline and/or lidocaine in an isotonic saline solution. Examples of infiltration solution are shown below.

TABLE 1
Infiltration Solution 1

| | Volume |
|---|---|
| Component | (mL) |
| Adrenaline stock solution | 2 |
| (to give a final concentration of 2 mg adrenaline per 1000 mL) | |
| 0.9% saline solution | 998 |
| | 1000 |

TABLE 2
Infiltration Solution 2

| | Volume |
|---|---|
| Component | (mL) |
| Adrenaline stock solution | 1 |
| (to give a final concentration of 1 mg adrenaline per 500 mL) | |
| Lidocaine stock solution | 20 |
| (to give a final concentration of 400 mg lidocaine per 500 mL) | |
| 8.4% sodium bicarbonate solution | 5 |
| 0.9% saline solution | 474 |
| | 500 |

### Devices and Systems

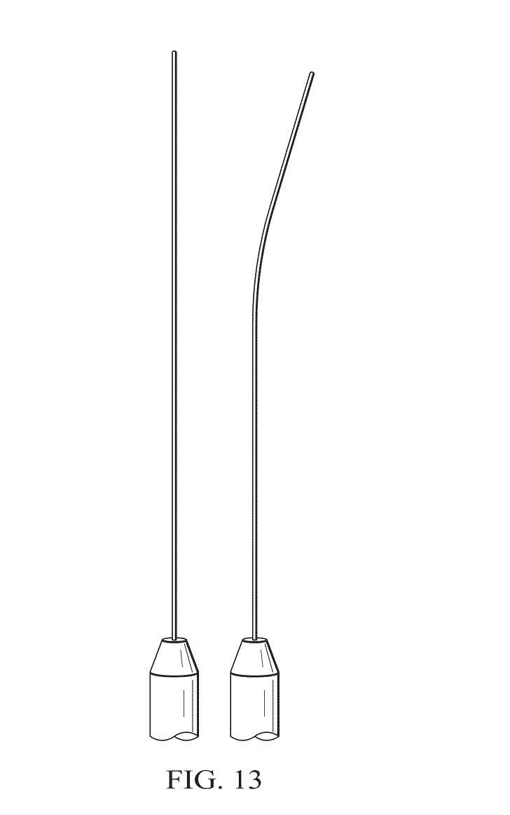

The present disclosure relates, in some embodiments, to apparatus for harvesting adipocytes. For example, the present disclosure provides a curved cannula. In some embodiments, a curved cannula may include an optical fiber configured to illuminate a tissue in a subject. A curved cannula may have an outside diameter of about 2 mm and a length from about 25 cm to about 30 cm according to some embodiments. A curve may be positioned anywhere along the length of a cannula and may occupy a single plane An example of a cannula is shown in FIG. 13. The left view of the example cannula is shown along the plane of the curvature and the right view is 90° from the plane of curvature.

According to some embodiments, the present disclosure relates to systems to aspirate and store fatty tissue for its later application in fat grafting and obtention of adipose-derived stem cells for means of cryopreservation and later clinical application. The present disclosure relates, in some embodiments, to systems for harvesting purifying cells (e.g., adipocytes, stem cells, fibroblasts) from subcutaeous tissues (e.g., human subcutaeous tissues). Systems may include, in some embodiments, kits of related materials and devices for assembly or use together in a cell harvesting purification method. Systems may form a single discrete unit or a plurality of interrelated units. For example, a system may comprise, according to some embodiments, a diode laser (e.g., a diode laser of 1210-nm wavelength), an optic fiber (e.g.,600-μm optical fiber) a cannula configured to convey light to a target site (e.g., a straight cannula and/or a curved cannula), a tissue aspirator (e.g., a straight or curved cannula), and/or a container to receive aspirated material. Some or all of these may be joined into a single unit and/or included in a kit for assembly by an intermediary or end user.

A system may comprise, according to some embodiments, a diode laser (e.g., a diode laser of 1210-nm wavelength) in optical communication with an optic fiber (e.g.,600-μm optical fiber), and/or a cannula configured to direct light (e.g., laser light) to a target site. In some embodiments, a system may include a cannula configured to receive a bodily fluid (e.g., a fluid comprising adipocytes), a fluid receptacle, a connector in fluid communication with the cannula and the receptacle, and a pump configured to apply a force or pressure that tends to move cells and/or fluid from the target site to the receptacle. According to some embodiments, at least a portion of a system or device of the disclosure may be configured to be sterile, sanitizable, disposable, replaceable, and/or repairable.

Systems and/or devices of the disclosure may be configured to assess and/or monitor a target site before, during, and/or after illumination and/or aspiration of cells. For example, a system may include and/or be configured to be compatible with an ultrasound apparatus. A system may include, in some embodiments, a viewing device configured to permit a health care worker to view the target site (e.g., either remotely or through a fiber linked to a light-delivery or tissue-removal cannula). A system may include, in some embodiments, a light emission detector, for example, to assess and/or monitor one or more aspects of the radiation delivered to the target site (e.g., wavelength, intensity). One or more displays may be connected with and/or included in a system. For example, one or more displays may be configured to show information about the subject (e.g., patient identification information, medical history), real-time and/or recorded images of the target site, system configuration and/or status information, and/or any other images or information of interest and/or use to the subject and/or practitioner.

According to some specific example embodiments, systems and/or devices may include a light source capable of emitting electromagnetic radiation of at least one wavelength. For example, systems and/or devices may include a light source that emits a broad or narrow band of wavelengths of infrared, visible, and/or ultraviolet light. The light source may also emit fluorescent or phosphorescent light. A light source may emit light continuously, intermittently and/or sporadically. In some specific example embodiments, systems and/or devices may include any additional spectrophotometry components including, without limitation, one or more modulators, polarizers, rhombs, etalons, prisms, windows, gratings, slits, interferometers, lenses, minors, reflective phase retarders, wavelength selectors, waveguides, beam expanders, beam splitters, and/or photodetectors.

A system may include one or more of the following components: a laser (e.g., a diode Laser of 1210-nm wavelength), a delivery cannula (e.g., a straight cannula of 2 mm outside diameter, 25 cm long for optic fiber; a curved cannula of 2 mm outside diameter, 25 cm long for optic fiber), a blade (e.g., a blade N° 11), a syringe (e.g., a syringe of 10 cc), a needle (e.g., a needle N° 21), a fluid-removal cannula (e.g., a cannula of 3 mm, straight and/or curved for aspiration; a cannula of 4 mm, straight and curved for aspiration), a syringe (e.g., a syringe of 60 cc), a receptacle (e.g., a sterile Recipient of 1000 cc to receive a bodily fluid), an infusion bag (e.g., an infusion Bag of up to 250 mm of Hg), a Klein needle (e.g., a Klein Needle 2 mm Outside Diameter (OD). 25 cm length; a Klein Needle 2.5 mm O.D., 25 cm length), and/or venoclysis Equipment

### Methods of Use

The first step is to determine the area to be treated. The surgeon must evaluate this area in order to obtain a minimum volume of 300 cc of fat. This technique requires IV sedation with local anesthesia or just local anesthesia depending on each patient.

The incision is marked with methylene blue. In this site, 2.5 mL of solution is infiltrated and a 2 mm incision is made with a N° 11 blade at the site determined in the pre-operative stage.

With the use of the Klein Needle local anesthesia is applied in the superficial layer (hypodermis). Next, 0.9% saline solution at 4° C. and the adrenaline 1:500,000 are administered. The super-wet technique is used: 1 L infiltrated per 1 L aspirated. A 150 mm Hg infusion bag is used, obtaining an approximate flow of 200 mL per minute through a 2.5 mm Klein Needle. The movements are performed slowly, allowing a proper expansion of the tissues to be treated. The infiltration of the cold solution is performed only in the deepest fat level, above the superficial fascia and within the adipose tissue; from here, the cold will spread to the entire thickness of the fat layer. Thus, the adipose tissue is preserved, achieving a good expansion on the site to be treated.

According to some embodiments, a laser may be used with a controller and a display that allows (e.g., through appropriate software) a user to monitor and/or adjust conditions including time, power (e.g., instantaneous, average, cumulative), heat (e.g., instantaneous, average, cumulative), and combinations thereof. A laser may be used in a continuous, repetitive, or other emission mode as desired. For example, a 1210-nm laser diode equipment, with a 600 micrometer optical fiber through a 2 mm O.D., 25 cm-long cannula (e.g., curved cannula, straight cannula) has been used by the present inventors, where the optical fiber must be 3 mm outside the tip of the cannula to permit the laser emission is 360°. Also a straight cannula (or a curved cannula, depending on the contour of the target area) in a continuous emission mode at 7 Watts is used. For each 10 cm² of area treated, with a thickness ranging between 3 and 4 cm of adipose tissue, this means that 315 J to 420 J are applied.

A method, in some embodiments, may include illuminating a tissue (e.g., a fat tissue) with a laser. Any suitable laser wavelength may be chosen. For example, a wavelength of light may be selected to achieve sufficient disruption of target tissue architecture. A wavelength from about 1150-nm to 1800-nm may be useful in some embodiments, because several these wavelengths provide high absorption/affinity with lipid-rich tissue. Similarly, any suitable intensity of illumination may be selected. In some embodiments, an intensity may be selected to achieve sufficient disruption of target tissue architecture. For example, illumination intensity (e.g., power applied) may range from about 4 Watts to about 4 Watts.

According to some embodiments, it may be desirable to harvest adipocytes under conditions that have as few adverse effects on the host subject as practicable. In some cases, for example, a method may include few or no unsafe conditions for the host subject. For example, the temperature at which method steps are performed may be monitored and/or controlled. A safe temperature range may be from about 27° C. to about 33° C. in some embodiments. Examples of safe temperatures are shown in Table 3.

TABLE 3
Safe Range of Temperatures is Tissues (1210 nm Laser)

Temperature (° C.)*

| Surgical Time | Skin | Subcutaneous Tissue |
|---|---|---|
| Pre-Infiltration | 31.5 | 33 |
| Post-Infiltration | 30 | 27 |
| Post-Laser | 31 | 30 |
| Post-Aspiration | 30 | 29.5 |

*Measured using an infrared thermometer for the skin temperature. The subcutaneous temperature was also measured as displayed in the chart.

The area monitored and/or maintained within a safe temperature range (e.g., the local area) may include subcutaneous tissue where a laser is applied in some embodiments. Temperatures outside a safe range may increase, according to some embodiments the risk of producing either hypothermia or excessive heat (burn) to the tissue.

A method may comprise, in some embodiments, a super-wet technique to infiltrate saline solution. Infiltration of saline solution may be performed at temperature intended to counter heat produced by laser emission. For example, a saline solution may be cold (e.g., ≦20° C. such as 4° C.) at the time of delivery In some embodiments, it may be desirable to balance the volume and temperature of the infiltration solution introduced to ameliorate and/or minimize tissue damage and/or distension. The ratio of infiltrated solution to aspirated fluid may be about 1:1 in some embodiments. The site of infiltration may be, according to some embodiments, the deepest layer of the subcutaneous tissue.

A laser may be applied with slow movements in some embodiments. Initially, it is applied to the deep layer, where cold solution has been infiltrated (to counterbalance the heat produced by the laser); it is then applied to the intermediate layer, to then finish at the superficial layer.

When the laser application is finalized, aspiration is performed by means of syringes and 3.5 to 4 mm, straight and curved cannulas with slow movements, as the liquefied fat allows easy aspiration. In some embodiments, aspiration is performed in the same sequence as the laser application. It starts on the deep layer (4 mm cannula) and ends in the superficial layer (3.5 mm cannula). Aspiration may be performed, according to some embodiments, using any aspiration device the surgeon considers suitable including, for example, a lipoaspirator machine and Coleman Systems®. The cannulas also may vary between 2 to 4 mm of outside diameter and from 15 cm to 30 cm of length.

The incisions are covered with Micropore® Tape, directly affixed to the skin, and the patient is covered with gauze and dressing pads. An elastic girdle, offering slight pressure, is provided to the patient in the operating room. Then, the patient is transferred to the recovery room, and will be discharged 2 to 3 hours later.

According to some embodiments, illumination (e.g., laser illumination) may be conveyed to tissue through any suitable light conveyance including, for example, an optical fiber, a light pipe, an optical cable, an optical waveguide, optical couplings, and combinations thereof. It may be desirable, according to some embodiments, to limit the distance from source to target site. One benefit that may arise from limiting this distance is that little or no loss in intensity may occur.

A light conveyance may be introduced to and/or moved through a subject's tissue to a target site using a catheter and/or cannula (e.g., about 2 mm in diameter). In some embodiments, a cold space is created (e.g., in and/or near the fluid-infiltrated tissue. A cold space may permit the appropriate and/or desired energy to be applied to the target site to cause selective photothermostimulation (SPS) which differs from well-know laser lipolysis. SPS allows users to apply a lser in a specific area only targeting certain structures (adipose tissue for example) without damaging other tissues. In some embodiments, tissue may be cooled to a temperature of from about 27° C. to about 33° C. (local temperature). Local temperature in this context may refer to tissue, for example, within about 10 cm of the target site.

Without limiting the disclosure to any particular mechanism of action, illuminating tissue according to some embodiments of the disclosure may create a disruption on the fat tissue's architecture, turning the solid fat into liquid, which is easier to aspirate. Adipocytes and/or stem cells may be aspirated (e.g., from fluid formed upon and/or after illumination) with little or no damage.

### Methods of Therapy

Adipocytes and stem cells purified and/or preserved according to some embodiments of the disclosure may be used in one or more medical/surgical applications with no trauma for the patient from whom the cells are harvested, which will be considered optimal and safe. Stem cells offer the possibility of a renewable source of replacement cells and tissues to treat diseases including Parkinson's Disease, Alzheimer's Disease, stroke, heart disease and diabetes mellitus. Also in the area of plastic surgery, stem cells may be used for generating new skin in the treatment of burned patients. Stem cells have been used in patients suffering from aniridia, a condition on which patients lose sight after chemical burns or genetic diseases. The treatment for aniridia is Limbal Stem Cell Therapy. By replacing the limbal stem cells the cornea begins to clear up as the cells are replaced with healthy transparent layer again.

As will be understood by those skilled in the art who have the benefit of the instant disclosure, other equivalent or alternative compositions, devices, methods, and systems for purifying and/or preserving cells can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the position and number of optical components may be varied. In some embodiments, a laser, an optical fiber, a cathether, and/or a cannula may be interchangeable. Interchageability may allow parts exposed to possible contaminants (e.g., cannulas) to be disposable while maintaining other components as reusable. In addition, the size of a device and/or system may be scaled up (e.g., to be used for adult subjects) or down (e.g., to be used for juvenile subjects) to suit the needs and/or desires of a practitioner. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/− about 10%, depicted value +/− about 50%, depicted value +/− about 100%) and/or a range endpoint. With respect to the former, a value of 50 depicted in an example, table, and/or drawing may form the basis of a range of, for example, about 45 to about 55, about 25 to about 100, and/or about 0 to about 100.

All or a portion of a device and/or system for purifying cells (e.g., adipocytes, stem cells) from mammalian tissues (e.g., adipose tissues) may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

### EXAMPLES

Some specific example embodiments of the disclosure may be illustrated by one or more of the examples provided herein.

### Example 1

### Adipocyte Purification

Liposuction in accordance with the present disclosure has been performed on 500 subjects. The procedures were performed using 980-nm and 1210-nm laser diode machines under local or regional anesthesia, as an ambulatory surgery with the exception of lipoabdominoplasty, which requires 24 hours of inpatient care. Safety parameters are based on the inventor's previous work with ex-vivo tissue (Abdominoplasty Flap), on which certain details for burn prevention were mentioned.

The following observations were made among treated patients:
Use of analgesics (maximum 48 hours), what demonstrates mild pain and a short recovery period;
Time to return to usual activities (average of 48 hours);
No major complications reported, like burns, infections, thromboembolism, fat embolism;
Minor complications reported included mild post-operative edema, ecchymosis lower than 2% of body surface area, and presence of skin retraction;
   Hystological and cytometric studies of the behavior of the fatty tissue treated with LASER were performed. These results demonstrate the security and effectiveness of this technique for body contouring surgeries.

### Example 2

### Mitochondrial Activity

For the study of adipocite viability, samples were evaluated by the Institute of Nutritional Research, Office of the University of Applied Sciences (Lima-Perú). After the Surgical procedure samples from the aspirated fat were studied and compared to samples from conventional liposuction. The values for mitochondrial activity were obtained through an MTT assay, which includes measuring the capacity of mitochondrial enzymes to reduce MTT (tetrazolium dye). Since only living cells are capable of reducing the dye, the number of living cells after a certain time, in this case 12 hours, can be assessed.

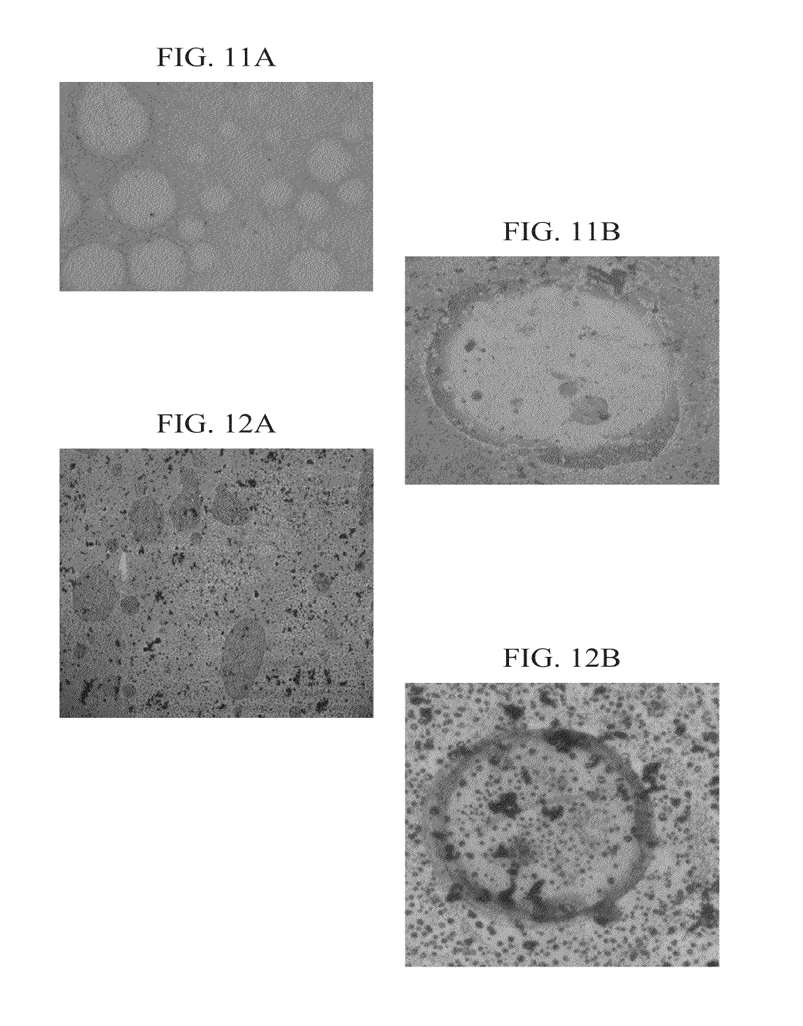

Mitochondrial activity measurements from post-procedure samples are shown in Table 4 and also in FIGS. 11-14. Samples “A” were obtained from Laser Liposuction and Samples “B” were obtained from conventional Liposuction. “Al” represents the group of samples studied at the same time as Group “B1”. “A2” represents the group of samples studied at the same time as Group “B2”. These results provide detail that laser liposuction is preferred to conventional liposuction since the number of viable cells obtained, for example, for fat grafting, is more than double.

TABLE 4
Mitochondrial Activity (ELISA Plate Reader)

| Samples | OD₄₅₀* | Procedure |
|---|---|---|
| A1 | 1.477 | Laser 1210-nm |
| A2 | 1.594 | Laser 1210-nm |
| B1 | 0.613 | Conventional liposuction |
| B2 | 0.658 | Conventional liposuction |
| MTT | 0.477 | Control |
| MTT | 0.400 | Control |

*MTT at 12 hours

What is claimed is:

1. A method for isolating cells from a subject, the method comprising:
contacting a deep layer of tissue with an aqueous infiltration fluid comprising saline and at least one pharmachologically active catecholamine prechilled to a temperature below 10° C. to form an infiltrated tissue;
illuminating a target site in the infiltrated tissue with light at a wavelength from about 1150-nm to about 1800-nm to produce at least a volume of liquified tissue comprising cells; and
aspirating at least some of the volume of liquified tissue comprising cells.

2. A method according to claim 1, wherein the subject is a human.

3. A method according to claim 1, wherein the cells are adipocytes or stem cells.

4. A method according to claim 1, wherein the wavelength of light is 1210-nm.

5. A method according to claim 1 further comprising monitoring the temperature of the target site.

6. A method according to claim 1 further comprising regulating the temperature of the target site.

7. A method according to claim 6, wherein the temperature is kept between about 27° C. and about 33° C.

8. A system for isolating cells from a subject, the system comprising:
a diode laser of 1210-nm wavelength;
a delivery cannula in optic communication with the diode laser and configured to illuminate a target site in a deep layer of the subject;
a fluid removal cannula configured to be positioned near the target site;
a fluid receptical in fluid communication with the fluid removal cannula; and
an aspiration pump operably coupled to the fluid removal cannula.

9. A method for harvesting adipocyte stem cells and adipocytes from a first subject for administration to a second subject, the method comprising:
contacting a deep layer of tissue with an aqueous infiltration fluid comprising saline and at least one pharmachologically active catecholamine prechilled to a temperature below 10° C. to form an infiltrated tissue;
illuminating a target site in the infiltrated tissue with light at a wavelength from about 1150-nm to about 1800-nm to produce at least a volume of liquified tissue comprising cells; and
aspirating at least some of the volume of liquified tissue comprising cells,
wherein the aspirated volume of liquified tissue comprises adipocyte stem cells and adipocytes.

10. A method according to claim 9 further comprising contacting a second subject actually or potentially in need of fat grafting or a regenerative medicine procedure with at least a portion of the aspirated volume of liquified tissue comprising adipocyte stem cells and adipocytes.

11. A method of selective photothermostimulation, the method comprising:
illuminating a subcutaneous target site with light from a diode laser at a wavelength of about 1210-nm through an optic fiber of about 600 μm,
wherein mitochondrial activity, cell proliferation or mitochondrial activity and cell proliferation in the illuminated subcutaneous target site is increased.
